(19) United States
(12) Patent Application Publication
(10) Pub. No.: US 2012/0058097 A1
(43) Pub. Date: Mar. 8, 2012

(54) COMPOSITIONS INCLUDING A SODIUM CHANNEL BLOCKER AND A B VITAMIN MOLECULE AND METHODS OF USE THEREOF

(75) Inventors: Michael J. Hudson, Berkshire (GB); Alan Michael Palmer, Berkshire (GB)

(73) Assignee: MS THERAPEUTICS LIMITED, Berkshire (GB)

(21) Appl. No.: 13/224,949

(22) Filed: Sep. 2, 2011

Related U.S. Application Data
(60) Provisional application No. 61/379,592, filed on Sep. 2, 2010.

(51) Int. Cl. A61K 31/519; A61P 25/18; A61P 25/28; A61P 25/04; A61K 31/675; A61P 25/00

(52) U.S. Cl. 424/94.1; 514/249; 514/81

(57) ABSTRACT

The invention generally relates to compositions including a sodium channel blocker and a B vitamin molecule and methods of use thereof. In certain embodiments, the invention provides compositions that include a sodium channel blocker and a B vitamin molecule. In other embodiments, the invention provides methods for treating a subject having a psychological disorder, a neurological disorder, a neurodengerative disorder, and/or a disorder associated with pain involving administering to the subject a sodium channel blocker and a B vitamin molecule.

### RELATED APPLICATION

The present application claims the benefit of and priority to U.S. provisional patent application Ser. No. 61/379,592, filed Sep. 2, 2010, the content of which is incorporated by reference herein in its entirety.

### FIELD OF THE INVENTION

The invention generally relates to compositions including a sodium channel blocker and a B vitamin molecule and methods of use thereof.

### BACKGROUND

Sodium channel blockers are compounds that impair conduction of sodium ions through sodium channels. Sodium channel blockers have been used to treat numerous types of disorders, such as psychological disorders, neurological disorders, neurodegenerative disorders, and disorders associated with pain. More recently, certain sodium channel blockers (e.g., lamotrigine and sipatrigine) has been found to be effective at treating multiple sclerosis. See Smith et al. (WO 99/52522), Harling et al. (WO 98/46574), Bountra et al. (WO 00/61231), Harbige et al. (U.S. 20040229873), and Kapoor et al. (The Lancet Neurology 9:681-688, 2010).

Multiple sclerosis affects over 400,000 people in the United States and approximately 2.5 million people worldwide. It is a chronic, auto-immune, inflammatory disease resulting in progressive demyelination and axonal loss within the central nervous system, leading to numerous physical and mental symptoms. The disease manifests itself through a wide variety of neurological symptoms that follow different patterns of evolution and variable rates of disability accumulation. The symptoms can be transient but, with disease progression, they become increasingly permanent. Symptoms include changes in sensation (reduced sensitivity and numbness), muscle weakness, muscle spasms, or difficulty in moving, difficulties with coordination and balance, problems in speech or swallowing, visual problems, fatigue and pain (both acute and chronic); along with bladder and bowel difficulties. Other features include cognitive impairment, of varying extent, as well as depression.

A side effect that has been observed during the treatment of multiple sclerosis with sodium channel blockers to treat multiple sclerosis is an apparently potentially reversible reduction in brain volume, described as pseudo-atrophy. See Kapoor et al. (The Lancet Neurology 9:681-688, 2010).

### SUMMARY

It is postulated that the reduction in brain volume associated with administration of certain sodium channel blockers to treat multiple sclerosis results from disturbances of folate metabolism in the brain caused by inhibition of dihydrofolate reductase brought about by the accumulation of the sodium channel blocker in the central nervous system. Inhibition of dihydrofolate reductase leads to imbalance of folate metabolism in the brain, resulting in reduction in brain volume, i.e., pseudo-atrophy. It has been discovered that pseudo-atrophy may be prevented by administering B vitamin molecules along with the sodium channel blocker, thereby compensating for any folate imbalance. Thus, administering a B vitamin molecule along with a sodium channel blocker may provide treatment for multiple sclerosis and any other disorder treated with a sodium channel blocker without the side effect of pseudo-atrophy.

Certain aspects of the invention provide compositions including a sodium channel blocker and a B vitamin molecule. Each of the sodium channel blocker and the B vitamin molecule is present in an effective dose. Exemplary sodium channel blockers include lamotrigine and compounds of the lamotrigine family. The B vitamin molecule may be any of the B vitamin molecules. Preferred B vitamin molecules are vitamin B9 molecules or analogs or derivatives thereof, such as tetrahydrofolate, methyl-tetrahydrofolate (CH₂-THF), and 10-formyl-tetrahydrofolate (10-CHO-THF). In certain embodiments, the tetrahydrofolate is a prodrug. An exemplary prodrug of tetrahydrofolate is a compound of Formula I:

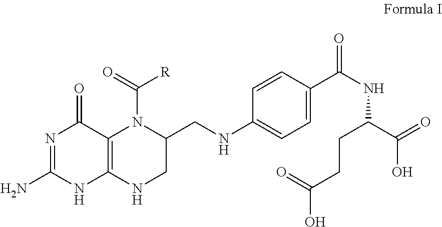

in which R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

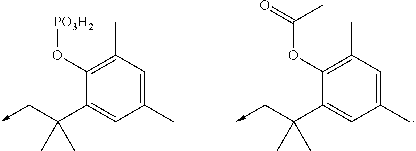

In particular embodiments, the sodium channel blocker is lamotrigine and the B vitamin molecule is a derivative of a vitamin B9 molecule that is selected from the group consisting of: tretrahydrofolate, methyl-tretrahydrofolate (CH₂-THF), and 10-formyl-tretrahydrofolate (10-CHO-THF). Compositions of the invention may further include a pharmaceutically acceptable buffer, and may be formulated as a unit dose.

Another aspect of the invention provides methods for treating a subject having a psychological disorder, a neurological disorder, a neurodengerative disorder, or pain related disorder, including administering to the subject a sodium channel blocker (e.g., lamotrigine or a compound of the lamotrigine family) and a B vitamin molecule. Preferred B vitamin molecules are vitamin B9 molecules or analogs or derivatives thereof, such as tetrahydrofolate, methyl-tetrahydrofolate (CH₂-THF), and 10-formyl-tetrahydrofolate (10-CHO-THF). In certain embodiments, the tetrahydrofolate is a prodrug. An exemplary neurodegenerative disorder is multiple sclerosis. The sodium channel blocker and the B vitamin molecule may be administered simultaneously or sequentially. In certain embodiments, the B vitamin molecule and the sodium channel blocker are administered at different frequencies.

Disorders to be treated include epilepsy, multiple sclerosis, glaucoma, uevetis, traumatic brain and spinal cord injury, cerebral ischemia, stroke and chronic neurodegenerative diseases, including Alzheimer's disease, frontotemporal dementia, Lewy body dementia, Parkinson's disease, Huntington's disease, motor neuron disease, as well as psychiatric disorders such as anxiety, bipolar disorder, mood disorders and schizophrenia and disorders involving pain, such as migraine, neuropathic pain and chronic pain.

Another aspect of the invention provides methods for reducing volume changes in a brain of a subject being treated for a disorder with a compound of the lamotrigine family including administering to a subject being treated with a compound of the lamotrigine family a B vitamin molecule.

### BRIEF DESCRIPTION OF THE DRAWINGS

FIG. 1 is a graph showing concentration of lamotrigine in rat brain following oral dosing.

### DETAILED DESCRIPTION

The invention generally relates to compositions including a sodium channel blocker and a B vitamin molecule and methods of use thereof. In certain aspects, the invention provides compositions including a sodium channel blocker and a B vitamin molecule.

### Sodium Channel Blockers

Voltage-gated sodium channels are complex membrane proteins that are widely expressed in neuronal, neuroendocrine, skeletal muscle and cardiac cells. These channels activate in response to membrane depolarization and, in most electrically excitable tissues, these channels are responsible for the rapid influx of sodium ions during a rising phase of an action potential.

A blocker of such sodium channels refers to a compound that impairs conduction of sodium ions through sodium channels. Under pathological conditions (such as ischaemia) sodium channels become abnormally activated resulting in an excessive flow of sodium ions into the cytoplasm. The rise in cellular sodium ions then causes a large inflow of calcium ions leading to the activation of several mechanisms that lead to irreversible loss of function and subsequent degeneration. It is possible to produce pharmacological agents capable of stopping excessive activity of sodium channels without adversely affecting their normal function. Indeed, this is the principal mode of action of several widely-used and well-tolerated antiepileptic drugs (e.g. phenyloin, carbamazapine and lamotrigine).

Sodium channel inhibitors have been shown to be protective towards neurons in the grey matter in several models of cerebral ischaemia. More recently, it has become evident that certain sodium channel inhibitors are highly effective in protecting axons in the in vitro optic nerve from irreversible damage imposed by severe deprivation of oxygen and glucose and in experimental models of spinal cord injury. (Stirling and Stys, Trends Mol. Med. 16:160-70, 2010). Blockers of voltage-gated sodium channels have proven therapeutic value in local anaesthesia, cardiac arrythmia, pain, bipolar disorder and epilepsy and are currently under investigation for stroke and other disorders. See e.g., Clare et al. (Drug Discov Today. 5: 506-520, 2000).

Sodium channel blockers that have been used to treat neurological and neurodegenerative disorders are shown in Smith et al. (WO 99/52522), Harling et al. (WO 98/46574), Bountra et al. (WO 00/61231), Harbige et al. (U.S. 20040229873), and Kapoor et al. (The Lancet Neurology 9:681-688, 2010), the content of each of which is incorporated by reference herein in its entirety. Exemplary disorders include epilepsy, multiple sclerosis, Alzheimer's disease, frontotemporal dementia, Lewy body dementia, Parkinson's disease, Huntington's disease, and motor neuron disease. Other neurological and neurodegenerative disorders that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Without being limited by any particular theory or mechanism of action, it is believed that in neurological and neurodegenerative disorders, axons succumb to damage through the loss of ionic homeostasis through sodium/potassium ATPase failure, sodium entry through persistent sodium channels and potassium efflux, causing the reversal of the sodium/calcium exchanger and a resultant accumulation of intra-axonal calcium. Many factors probably play a role in this process, including nitric oxide (NO). It has previously been shown that axons exposed to NO in vivo can undergo degeneration, especially if the axons are electrically active during NO exposure (Kapoor et al., The Lancet Neurology 9:681-688, 2010; and Stirling and Stys, Trends Mol. Med. 16:160-70, 2010). The axons may degenerate because NO can inhibit mitochondrial respiration, leading to intraaxonal accumulation of Na⁺ and Ca²⁺ ions. It has been found that axons can be protected from NO-mediated damage using Na⁺ channel blockers (Kapoor et al., The Lancet Neurology 9:681-688, 2010).

Sodium channel blockers that have been used to treat psychiatric disorders are shown in Stahl (J Clin Psychiatry, 65(6):738-739, 2004). Exemplary disorders include anxiety, bipolar disorder, mood disorders and schizophrenia. Other psychiatric disorders that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Sodium channel blockers that have been used to treat disorders involving pain are shown in Bhattacharya (Neurotherapeutics, 6(4):663-678, 2009). Exemplary disorders include migraine, neuropathic pain, and chronic pain. Other disorders involving pain that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Sodium channel blockers have also been used to treat stroke (Hewitt et al., Brain Res., 898(2):281-287, 2001), glaucoma (Haim et al., Invest Ophthalmol V is Sci., 46(11):4164-4169, 2005), uveitis (U.S. Pat. No. 6,221,887), traumatic brain and spinal cord injury (Schwartz et al., J. Neurosurg., 94(2 Suppl):245-256, 2001), and cerebral ischemia (Hewitt et al., Brain Res., 898(2):281-287, 2001). The content of each of these references is incorporated by reference herein in its entirety.

In certain embodiments, the sodium channel blocker is a compound of the lamotrigine family of compounds. Such compounds include analogs, derivatives, salts, and prodrugs of lamotrigine. Exemplary compounds of the lamotrigine family are shown in Harbige et al. (U.S. 20040229873), the content of which is incorporated by reference herein in its entirety. Exemplary compounds include lamotrigine; 3,5-diamino-6-(2,3-dichlorophenyl)-1,2,4-triaz-ine; Sipatrigine; 4-amino-2-(4-methyl-1-piperazinyl)-5-(2,3,5-trichlorophe-nyl)-pyrimidine; 2,4-diamino-5-(2,3-dichlorophenyl)-6-(fluoromethylpyrimid-ine); R-(−)-2,4-diamino-6-fluoromethyl-5-(2,3,5-trichlorophenyl)-pyrimidin-e; 4-amino-2-(1-piperazinyl)-5-(2,3,5-trichlorophenyl)-pyrimidine (active Sipatrigine metabolite); 4-amino-2-(4-methyl-1-piperazinyl)-5-(2,3,5-trichlorophenyl)-6-trifluoromethylpyrimidine; 2,4-diamino-5-(2,3,5-trichloroph-enyl)-trifluoromethylpyrimidine; 2,4-diamino-5-(2,3,5-trichlorophenyl)-6-m-ethoxymethylpyrimidine; 4-amino-6-methyl-2-(4-methyl-1-piperazinyl)-5-(2,3-,5-trichlorophenyl)-pyrimidine; 4-amino-2-(4-propyl-1-piperazinyl)-5-(2,3,-5-trichlorophenyl)-pyrimidine; and 2,4-diamino-5-(2,3,5-trichlorophenyl)-py-rimidine. Several of these compounds are described in U.S. Pat. Nos. 5,635,507, 5,597,828, 5,684,005, 5,587,380, 5,712,276 and 5,712,277 all of which are incorporated herein by reference.

A general review of pro-drugs can be found in Nature Reviews Drug Discovery 7:255-270, 2008. A specific review of prodrugs of parent drugs bearing an amino group is found in Molecules 13:519-547, 2008. Some examples of prodrug moieties in the literature that have been used to modulate the stability and pharmacokinetic properties of active drug molecules bearing one or more amino groups are: natural amino acids, attached through the carboxyl group (e.g. Current Medicinal Chemistry, 11:1241-1253, 2004; International Journal of Pharmaceutics 121:157-167, 1995; WO 2005/046575A2; J. Med. Chem., 45:744, 2002; Eur. J. Med. Chem., 26:143, 1991), carbamates (U.S. Pat. No. 7,060,259; U.S. Pat. No. 5,401,868; J. Med. Chem., 47:2651, 2004), phosphates and phosphonate esters (J. Org. Chem., 61:8636, 1996; Bioorg. and Med. Chem. Lett., 8:3159, 1998), sulphamic acids (Bioorg & Med. Chem. Lett., 11:1093, 2001), esters (Pharmaceutical Research, 8:455, 1991), amides (J. Med. Chem., 43:2530, 2000) and azo derivatives (Am. J. Gastroenterol., 97:2939, 2002), the content of each of which is incorporated by reference herein in its entirety. All the prodrugs can be synthesised by one skilled in the art, using an appropriate modification of the formylation process described by Sata et al. (Analytical Biochemistry 154:516-524, 1986).

In certain embodiments, the sodium channel blocker is lamotrigine, carbamazepine, oxcarbazepine, valproic acid, sipatrigine, 4030w92, 202w92, 78c90 (active sipatrigine metabolite), 440c89, 149C89, 722c90, 279c90 or 1003c87. In particular embodiments, the sodium channel blocker is lamotrigine. Lamotrigine is commercially available from GlaxoSmithKline under the product name LAMICTAL, and is described in Bountra et al. (WO 00/61231).

### B Vitamin Molecules

A “B vitamin molecule”, as used herein, refers to any or all of a complex of several vitamins that were discovered during early studies of human nutrition, exemplified by vitamin B1 (thiamine), vitamin B2 (riboflavin), vitamin B3 (vitamin P or vitamin PP, or niacin), vitamin B5 (pantothenic acid), vitamin B6 (pyridoxine and pyridoxamine), vitamin B7 (vitamin H, vitamin B-w, or biotin), vitamin B9 (vitamin M, vitamin B-c, or folic acid), and vitamin B12 (cyanocobalamin).

A B vitamin molecule also includes without limitation, “nonhuman forms” discovered by study of nutrition in other life form (animals, bacteria, yeast, etc.) such as vitamin B4 (adenine), vitamin B8 (ergadenylic acid), vitamin B10 (para-aminobenzoic acid), vitamin B11 (salicylic acid or vitamin S), vitamin B13 (pyrimidinecarboxylic acid or orotic acid), vitamin B14 (a mixture of vitamin B10 and vitamin B11), vitamin B15 (pangamic acid or dimethylglycine), vitamin B16, vitamin B17 (amygdalin), vitamin B22, vitamin B-t (L-carnitine), and vitamin B-x (para-aminobenzoic acid).

The B vitamins often work together to deliver a number of health benefits to the body, such as, bolstering metabolism, maintaining healthy skin and muscle tone, enhancing immune and nervous system function, and promoting cell growth and division. Combined, the B vitamins assist in combating the symptoms and causes of stress, depression, and cardiovascular disease. B vitamins are water soluble and are dispersed throughout the body and must be replenished daily, as any excess is excreted generally in the urine.

A “vitamin B2 molecule”, as used herein, refers to any or all of vitamin B2, riboflavin or vitamin G. As used herein, this term includes also the coenzyme forms, flavin adenine dinucleotide (FAD) and flavin adenine mononucleotide (FMN). B2 molecules are easily absorbed, water-soluble micronutrients that support energy production by aiding in the metabolism of fats, carbohydrates, and proteins. Vitamin B2 molecules are also needed for red blood cell formation and respiration, antibody production, and for regulating human growth and reproduction. They function as antioxidants by scavenging damaging particles in the body known as free radicals. Vitamin B2 molecules are important for healthy skin, nails, hair growth and general good health, including regulating thyroid activity.

Vitamin B2 deficiency manifests itself as cracks and sores at the corners of the mouth, eye disorders, inflammation of the mouth and tongue, skin lesions, dermatitis, dizziness, hair loss, insomnia, light sensitivity, poor digestion, retarded growth, and the sensation of burning feet.

An exemplary structure of the vitamin B2 molecule is shown below:

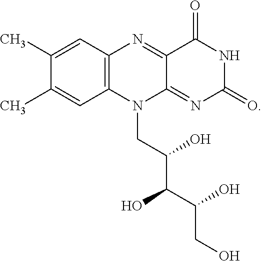

A “vitamin B3 molecule”, as used herein, refers to any or all of vitamin B3, niacin, or nicotinic acid. These include the amide form, nicotinamide or niacinamide. Vitamin B3 molecules are water-soluble vitamins whose derivatives such as NADH, NAD, NAD⁺, and NADP play important roles in energy metabolism in the living cell and DNA repair. These molecules also assist the body make various sex and stress-related hormones in the adrenal glands and other parts of the body. A vitamin B3 molecule is effective in improving circulation and reducing cholesterol levels in the blood.

Lack of a vitamin B3 molecule causes the deficiency disease pellagra. A mild B3 deficiency causes a slow down of the metabolism, which in turn causes a decrease in cold tolerance and is a potential contributing factor towards obesity.

In vivo synthesize a vitamin B3 molecule is initiated from the 5-membered aromatic heterocycle of the amino acid tryptophan, which is cleaved and rearranged with the alpha amino group of tryptophan into the 6-membered aromatic heterocycle of a vitamin B3 molecule. The reaction proceeds as follows: tryptophan->kynurenine->3-hydroxy kynurenine (B6 enzyme needed)->vitamin B3 molecule. The liver can synthesize vitamin B3 molecules from the amino acid tryptophan, and the synthesis is slow and requires vitamin B6, i.e., 60 mg of tryptophan are required to make one milligram of a vitamin B3 molecule. An exemplary structure of the vitamin B3 molecule is shown below:

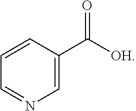

A “vitamin B6 molecule”, as used herein, refers to any or all of vitamin B6, pyridoxine, pyridoxal, and pyridoxamine. These molecules are converted to pyridoxal 5′-phosphate (PLP) in the liver. PLP is an important cofactor for numerous metabolic enzymes, such as aminotransferases, amino acid racemases, and amino acid decarboxylases, most of which have amino group-containing compounds as substrates. In the absence of PLP, a substantial number of cellular biosynthetic and catabolic pathways would cease to function.

Two pathways of de novo PLP synthesis are known, the PdxA/PdxJ pathway and the PDX1/PDX2 pathway. Organisms appear to contain either one or the other pathway of de novo PLP synthesis. Vitamin B6 comprises, in addition to PLP, precursors of PLP in phosphorylated and non-phosphorylated forms, and these compounds are referred to as B6 vitamers. Non-phosphorylated vitamers pyridoxine, pyridoxal and pyridoxamine can be taken up by many bacteria, fungi, plants, and mammalian cells and converted into PLP by a salvage pathway. An exemplary structure of the vitamin B6 molecule is shown below:

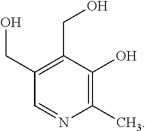

A “vitamin B9 molecule”, as used herein, refers to any or all vitamin B9, folic acid and folate. The B9 molecule is a water-soluble vitamin that is important for the production and maintenance of new cells, particularly during periods of rapid cell division and growth such as infancy and pregnancy. The B9 molecule is needed to replicate DNA and synthesize RNA, and is involved in the synthesis, repair, and functioning of DNA. A deficiency of folate may result in damage to DNA that may lead to cancer. Both adults and children need vitamin B9 molecules to make normal red blood cells and prevent anemia.

Signs of vitamin B9 deficiency include diarrhea, loss of appetite, weight loss, weakness, sore tongue, headaches, heart palpitations, irritability, and behavioral disorders. In adults, anemia is a sign of advanced vitamin B9 deficiency. In infants and children, vitamin B9 deficiency can slow growth rate.

An exemplary structure the vitamin B9 molecule is shown below:

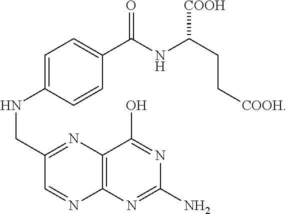

A “vitamin B12 molecule”, as used herein, refers to any or all of a group of cobalt containing tetrapyrrole compounds known as corrinoids. Examples include, cobalamin, cyanocobalamin, hydroxocobalamin, and thiocyanate cobalamin. The structure of vitamin B12 molecules comprises a nucleotide (base, ribose and phosphate) attached to a corrin ring which is made up of four pyrrole groups and an atom of cobalt in the center. The cobalt atom bonds to a methyl group, a deoxyadenosyl group, and a hydroxyl group or a cyano group. A vitamin B12 molecule includes the coenzyme forms of vitamin B12, i.e., methylcobalamin and 5-deoxyadenosylcobalamin (adenosylcobalamin).

Deficiency of vitamin B12 results in hematological, neurological and gastrointestinal effects. The hematological effects are caused by interference with DNA synthesis. The hematologic symptoms and signs of vitamin B12 deficiency, include hypersegmentation of polymorphonuclear leukocytes, macrocytic, hyperchromic erythrocytes, elevated mean corpuscular volume (MCV), elevated mean corpuscular hemoglobin concentration (MCH, MCHC), a decreased red blood cell count, pallor of the skin, decreased energy and easy fatigability, shortness of breath and palpitations.

The neurological effects of the vitamin B12 deficiency include tingling and numbness in the extremities (particularly the lower extremities), loss of vibratory and position sensation, abnormalities of gait, spasticity, Babinski's responses, irritability, depression and cognitive changes (loss of concentration, memory loss, dementia). Visual disturbances, impaired bladder and bowel control, insomnia and impotence may also occur.

Gastrointestinal effects of vitamin B12 deficiency include intermittent diarrhea and constipation, abdominal pain, flatulence and burning of the tongue (glossitis). Anorexia and weight loss are general symptoms of vitamin B12 deficiency. Pathologies or defects can reduce efficiency or function of this pathway, such as an autoimmune condition involving formation of antibodies against the cells producing intrinsic factor; presence of a fish tapeworm; or the after-effects of surgery to the small intestine which results in the surface of the small intestine being insufficient to obtain B12 and intrinsic factor. These pathologies or defects result in less efficient absorption of vitamin B12, and could be ameliorated by administration of a higher dosage of vitamin B12.

An exemplary structure of a vitamin B12 molecule is shown below:

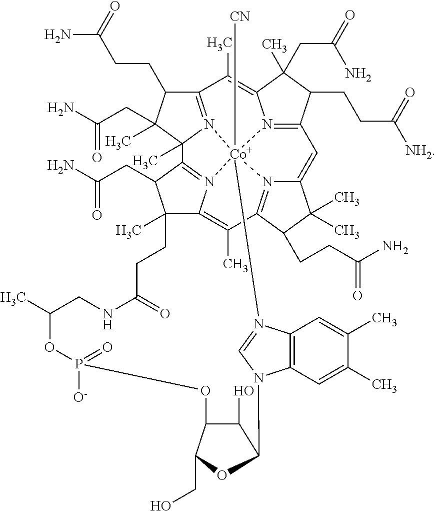

In certain embodiments, the B vitamin molecule is a vitamin B9 molecule, which includes, salts, analogs, derivatives, or prodrugs thereof. In particular embodiments, the B vitamin is a derivative of a vitamin B9 molecule. Exemplary derivatives of vitamin B9 molecules include tetrahydrofolate, methyl-tetrahydrofolate (CH₂-THF), and 10-formyl-tetrahydrofolate (10-CHO-THF).

In the form of a series of tetrahydrofolate compounds, folate derivatives are coenzymes in a number of single carbon transfer reactions biochemically, and also is involved in the synthesis of dTMP (2′-deoxythymidine-5′-phosphate) from dUMP (2′-deoxyuridine-5′-phosphate).

The pathway in the formation of tetrahydrofolate (Fe) is the reduction of folate (F) to dihydrofolate (FH₂) by folate reductase, and then the subsequent reduction of dihydrofolate to tetrahydrofolate (FH₄) by dihydrofolate reductase. Methylene tetrahydrofolate (CH₂FH₄) is formed from tetrahydrofolate by the addition of methylene groups from one of three carbon donors: formaldehyde, serine, or glycine. Methyl tetrahydrofolate (CH₃—FH4) can be made from methylene tetrahydrofolate by reduction of the methylene group, and formyl tetrahydrofolate (CHO—FH₄, folinic acid) is made by oxidation of the methylene tetrahydrofolate.

THF is the folate product of the methionine synthase reaction, in addition to the reduction of folic acid. 5-Methyltetrahydrofolate is generated by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase. 5-Methyltetrahydrofolate donates a methyl group to homocysteine, in conversion of homocysteine to L-methionine. The enzyme that catalyzes the reaction is methionine synthase. Vitamin B₁₂ is a cofactor in the reaction. This reaction, in which vitamin B₁₂ is a cofactor, is of great importance in the regulation of serum homocysteine concentration. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as for the synthesis of epinephrine, melatonin, creatine and other molecules. Tetrahydrofolate is the folate product of the methionine synthase reaction. 5-0-Methylenetetrahydrofolate, in addition to its role in the metabolism of homocysteine, supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme DHFR.

Folinic acid is a 5-formyl derivative of THF that is readily converted to tetrahydrofolate and thus has vitamin activity which is equivalent to folic acid. Leucovorin is a commercially available agent that is a mixture of diastereoeisomers of folinic acid. The biologically active compound of the mixture is the (−)-1-isomer, known as citrovorum factor or (−)-folinic acid. Leucovorin does not require reduction by the enzyme DHFR in order to participate in reactions utilizing folates, principally one-carbon transfer reactions. Administration of leucovorin can counteract the therapeutic and toxic effects of folic acid antagonists such as methotrexate, which act by inhibiting dihydrofolate reductase. Folinic acid is available as a calcium salt for parenteral or oral administration.

Leucovorin is used as an antidote to drugs which act as DHFR inhibitors. Leucovorin is employed in injection form as leucovorin calcium in an aqueous bacteriostatic preparation containing leucovorin present as the calcium salt pentahydrate of N-[4-[[(2-amino-5-formyl-1,4,5,6,7,8-hexahydro-4-oxo-6-pteri-dinyl)-methyl-]amino-]benzoyl]-L-glutamic acid. Each 5 mg of leucovorin is equivalent to 5.4 mg of anhydrous leucovorin calcium or 6.35 mg of leucovorin calcium pentahydrate.

In other embodiments, the B vitamin molecule is a prodrug of tetrahydrofolate. Methods of making prodrugs are described above. Exemplary prodrugs of tetrahydrofolate include the general structure of tetrahydrofolate having certain prodrug moieties attached at the 5-position include the following: amides, including natural and unnatural amino acid amides; carbamates; aryl phosphonic acids; and aryl esters. An exemplary prodrug of tetrahydrofolate is shown as Formula I below:

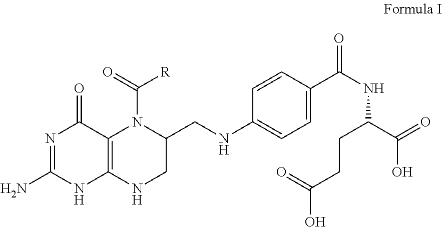

wherein R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

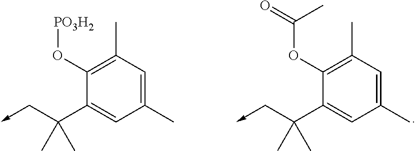

### B Vitamin Dosage

A B vitamin molecule is administered systemically, for example, orally, subcutaneously, intramuscularly, and intravenously. The dose of B vitamin administered depends on form and route of delivery, i.e., injection, nasal gel, or oral administration by lozenges or by sublingual tablets, as is well known to one of ordinary skill in the art of nutritional supplementation. As B vitamins are water soluble and as excess is not stored but is excreted generally in the urine, it is important that they are replenished daily. Table 1 below shows typical dosages of different B vitamins.

TABLE 1
Dosages of different B vitamins

| | | | | Potential |
|---|---|---|---|---|
| | Minimum | Typical | Upper | side ef- |
| | recommended | therapeutic | limit of | fects at |
| Vita- | daily allow- | daily | daily | upper |
| min | ance (MDR) | dose | intake | limits |
| B2 | 1 mg-2 mg | 50 mg-100 mg | N/A | No known |
| | | | | toxicity |
| B3 | 15 mg-25 mg | 100 mg-500 mg | Above 3000 mg | Liver |
| | | | | problems |
| B6 | 1.5 mg-2.5 mg | 50 mg-100 mg | Above 100 mg | Numbness |
| | | | | and tin- |
| | | | | gling in |
| | | | | the fingers |
| | | | | and toes |
| B9 | 350 μg-450 μg | 500 μg-1000 μg | N/A | No known |
| | | | | toxicity |
| B12 | 3 μg-30 μg | 500 μg-5000 μg | N/A | No known |
| | | | | toxicity |

The amount of total absorption of these B vitamins increases with increased intake. Without being limited by any particular theory or mechanism of action, higher doses than the minimum daily requirement are beneficial under circumstances of vitamin stress, such as during a treatment regimen. Excess amounts of B vitamins that are administered are subsequently excreted in the feces and in the urine. In general, if the circulating levels of the B vitamins exceed the B vitamin binding capacity of the blood, the excess is excreted in the urine.

Use of a B Vitamin Molecule with a Sodium Channel Blocker

It has been found that the reduction in brain volume associated with administration of sodium channel blockers results from disturbances of folate metabolism in the brain caused by inhibition of dihydrofolate reductase brought about by the accumulation of the sodium channel blocker in the central nervous system. Inhibition of dihydrofolate reductase leads to imbalance of folate metabolism in the brain, resulting in reduction in brain volume, i.e., pseudo-atrophy. It has been discovered that pseudo-atrophy may be prevented by administering B vitamin molecules along with the sodium channel blocker, thereby compensating for any folate imbalance. Thus, administering a B vitamin molecule along with a sodium channel blocker provides treatment for disorders treated with a sodium channel blocker without the side effect of pseudo-atrophy.

In a particular embodiment, a B vitamin is used in combination with lamotrigine for treatment of multiple sclerosis. On the basis of a study 120 people with secondary progressive multiple sclerosis, lamotrigine appears to show evidence of neuroprotective efficacy as it was shown to approximately halve the deterioration of walking speed compared to the control group Kapoor et al. (The Lancet Neurology 9:681-688, 2010). However, MRI measurements of brain volume were the primary outcome measure in this study and this revealed an early reduction in brain volume which was reversible over the 2 year course of the trial.

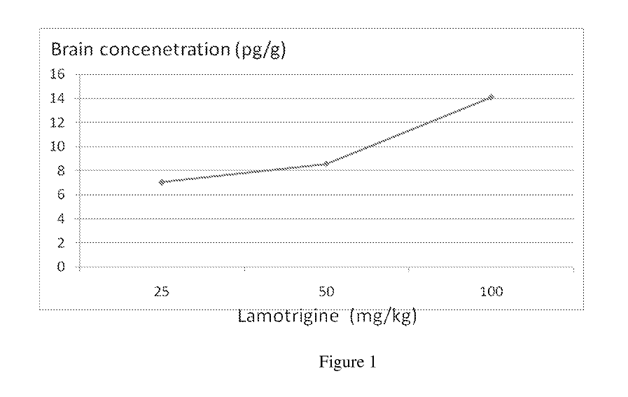

Without be limited by any particular theory or mechanism of action, it is believed that the pseudo-atrophy and a gait disturbance are attributable to disturbances of folate metabolism in the brain caused by inhibition of dihydrofolate reductase brought about by the accumulation of lamotrigine in the central nervous system, which occurs as a result of its long half life (23-36 h) and its inhibition of the efflux transporter P-glycoprotein. Thus, lamotrigine blocks its own egress from the central nervous system, as illustrated by the data in FIG. 1.

Therefore, it is believed that lamotrigine-induced pseudoatrophy observed by MRI studies of people with multiple sclerosis, is attributable to its inhibition of dihydrofolate reductase and the subsequent imbalance of folate metabolism in the brain. On this basis, pseudo-atrophy can be prevented by correcting any folate imbalance using compounds such as B vitamin molecules, particularly vitamin B9 molecules, and more particularly derivatives of vitamin B9 molecules such as tetrahydrofolate, methyl-tetrahydrofolate (CH₂-THF), and 10-formyl-tetrahydrofolate (10-CHO-THF).

### Pharmaceutical Compositions

The sodium channel blockers and B vitamin molecules of the pharmaceutical compositions, uses, and methods described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The sodium channel blockers herein may be used, for example, for the preparation of pharmaceutical compositions that comprise an effective amount of a sodium channel blocker herein and a B vitamin molecule, or a pharmaceutically acceptable salt thereof, as active ingredient together or in admixture with a significant amount of one or more inorganic or organic, solid or liquid, pharmaceutically acceptable carriers.

The compositions herein are suitable for administration to a warm-blooded animal, including, for example, a human (or to cells or cell lines derived from a warm-blooded animal, including for example, a human cell), for the treatment or, in another aspect of the invention, prevention of (also referred to as prophylaxis against) a disease that responds to inhibition of sodium channel activity, comprising an amount of a compound of the present methods or a pharmaceutically acceptable salt thereof, which is effective for this inhibition, together with at least one pharmaceutically acceptable carrier, and a B vitamin molecule.

The pharmaceutical compositions according to the methods are those for enteral, such as nasal, rectal or oral, or parenteral, such as intramuscular or intravenous, administration to warm-blooded animals (including, for example, a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The dose of a sodium channel blocker of the present methods or a pharmaceutically acceptable salt thereof to be administered to warm-blooded animals, for example humans of approximately 70 kg body weight, is for example, from approximately 3 mg to approximately 10 g, from approximately 10 mg to approximately 1.5 g, from about 100 mg to about 1000 mg/person/day, divided into 1-3 single doses which may, for example, be of the same size. Usually, children receive half of the adult dose.

The dose of the B vitamin molecule to be administered to warm-blooded animals, for example humans of approximately 70 kg body weight, is for example at least about 50 micrograms (μg), at least about 80 μg, 90 μg, 100 μg, or at least about 500 μg, at least about 25 milligrams (mg), 30 mg, 40 mg, or at least about 50 mg, to at least about 500 mg.

The pharmaceutical compositions have from approximately, for example, 1% to approximately 95%, or from approximately 20% to approximately 90%, active ingredients. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredients, and also suspensions, and especially isotonic aqueous solutions or suspensions, are used, it being possible, for example in the case of lyophilized compositions that have the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may have viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned, for example, liquid fatty acid esters that contain as the acid component a long-chained fatty acid having from 8-22, or from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, .beta.-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C8 to C12, Huls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredients with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Suitable carriers are for example, fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredients in the form of granules, for example with fillers, such as lactose; binders, such as starches, and/or glidants, such as talc or magnesium stearate, and if desired with stabilizers. In soft capsules the active ingredients are preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragee coatings or the capsule casings, for example for identification purposes or to indicate different doses of active ingredient.

The pharmaceutical compositions generally include an effective dose of each of the sodium channel blocker and the B vitamin molecule. As used herein, an “effective dose” means an amount of each active component that is different from an optimal amount of that component if administered in a therapeutic regiment absent the other active component. An effective dose of the pharmaceutical composition when administered to a subject, prevents or ameliorates a disease symptom also produces fewer side effects compared to these symptoms in a control subject administered either the sodium channel blocker or the B vitamin molecule alone. One of ordinary skill in the art can readily determine an effective amount of each component in the combination. For example, side effects are prevented or ameliorated by the presence in the combination of a particular dose of B vitamin, then a greater amount of the sodium channel blocker component can be included in the pharmaceutical composition to be administered to the subject, compared to a control amount, which is the amount of the sodium channel blocker alone that would be administered to the subject. It is an object of the methods and compositions herein that in the presence or co-administration of a B vitamin, an effective dose of an sodium channel blocker is reduced compared to an effective dose in the absence of a B vitamin, due to increased efficacy of these compounds in the presence of the B vitamin.

Under certain conditions, the B vitamin molecules have a synergistic effect in combination with a class of sodium channel blockers, in preventing or ameliorating a disease that responds to inhibition of sodium channel activity. Thus the pharmaceutical composition includes an effective dose which is a lesser amount of the sodium channel blocker component, compared to administering to the subject the sodium channel blocker alone, to obtain a comparable therapeutic effect.

An effective dose of the B vitamin component of the pharmaceutical composition is an amount that prevents or ameliorates one or more side effects resulting from administration of an sodium channel blocker, and is described herein.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A composition comprising a sodium channel blocker and a B vitamin molecule.

2. The composition according to claim 1, wherein each of the sodium channel blocker and the B vitamin molecule is present in an effective dose.

3. The composition according to claim 1, further comprising a pharmaceutically acceptable buffer.

4. The composition according to claim 1, wherein the composition is in a unit dose.

5. The composition according to claim 1, wherein the sodium channel blocker is a compound of the lamotrigine family.

6. The composition according to claim 5, wherein the compound is lamotrigine.

7. The composition according to claim 1, wherein the B vitamin molecule is a vitamin B9 molecule or an analog or derivative thereof.

8. The composition according to claim 7, wherein the derivative of the vitamin B9 molecule is selected from the group consisting of: tetrahydrofolate, methyl-tetrahydrofolate (CH₂-THF), and 10-formyl-tetrahydrofolate (10-CHO-THF).

9. The composition according to claim 8, wherein the tetrahydrofolate is a prodrug.

10. The composition according to claim 9, wherein the prodrug is a compound according to Formula I: 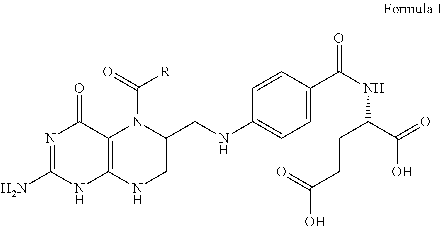
wherein R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

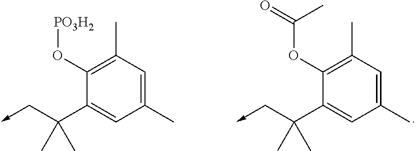

11. The composition according to claim 1, wherein the sodium channel blocker is lamotrigine and the B vitamin molecule is a derivative of a vitamin B9 molecule that is selected from the group consisting of: tretrahydrofolate, methyl-tretrahydrofolate (CH₂-THF), and 10-formyl-tretrahydrofolate (10-CHO-THF).

12. A method for treating a subject having a psychological disorder, neurological disorder, neurodengerative disorder, and/or a disorder associated with pain, the method comprising: administering to the subject a sodium channel blocker and a B vitamin molecule.

13. The method according to claim 12, wherein the subject is a human.

14. The method according to claim 12, wherein the sodium channel blocker is a compound of the lamotrigine family.

15. The method according to claim 14, wherein the compound is lamotrigine.

16. The method according to claim 12, wherein the B vitamin molecule is a vitamin B9 molecule or an analog or derivative thereof.

17. The method according to claim 16, wherein the derivative of the vitamin B9 molecule is selected from the group consisting of: tetrahydrofolate, methyl-tetrahydrofolate (CH₂-THF), and 10-formyl-tetrahydrofolate (10-CHO-THF).

18. The method according to claim 17, wherein the tetrahydrofolate is a prodrug.

19. The method according to claim 18, wherein the prodrug is a compound according to Formula I: 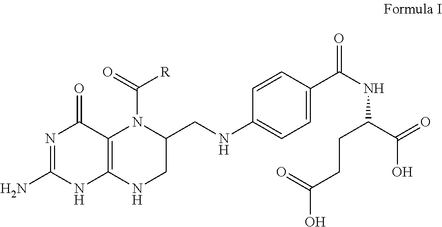
wherein R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

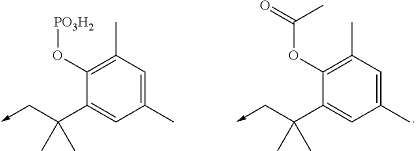

20. The method according to claim 12, wherein the sodium channel blocker is lamotrigine and the B vitamin molecule is a derivative of a vitamin B9 molecule that is selected from the group consisting of: tretrahydrofolate, methyl-tretrahydrofolate (CH₂-THF), and 10-formyl-tretrahydrofolate (10-CHO-THF).

21. The method according to claim 12, wherein administering is simultaneous.

22. The method according to claim 12, wherein administering is sequential.

23. The method according to claim 12, the vitamin and the sodium channel blocker are administered at different frequencies.

24. The method according to claim 12, wherein the neurodegenerative disease is multiple sclerosis.

25. A method for reducing volume changes in a brain of a subject being treated for a disorder with a compound of the lamotrigine family, the method comprising: administering to a subject being treated with a compound of the lamotrigine family a B vitamin molecule.

26. The method according to claim 25, wherein the B vitamin molecule is a vitamin B9 molecule or an analog or derivative thereof.

27. The method according to claim 26, wherein the derivative of the vitamin B9 molecule is selected from the group consisting of: tretrahydrofolate, methyl-tretrahydrofolate (CH₂-THF), and 10-formyl-tretrahydrofolate (10-CHO-THF).

28. The method according to claim 27, wherein the tetrahydrofolate is a prodrug.

29. The method according to claim 28, wherein the prodrug is a compound according to Formula I: 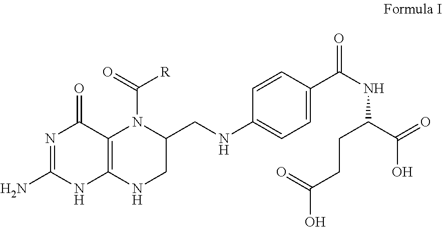
wherein R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

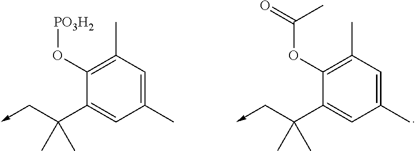

30. The method according to claim 25, wherein administering the B vitamin molecule is simultaneous with administration of the compound of the lamotrigine family.

31. The method according to claim 25, wherein administering the B vitamin molecule is sequential to administration of the compound of the lamotrigine family.

32. The method according to claim 25, wherein the B vitamin and the compound of the lamotrigine family are administered at different frequencies.

33. The method according to claim 25, wherein the disorder is selected from the group consisting of a psychological disorder, a neurological disorder, a neurodegenerative disorder, and a disorder involving pain.

34. The method according to claim 33, wherein the neurodegenerative disorder is multiple sclerosis.

35. The method according to claim 25, wherein the compound of the lamotrigine family is lamotrigine.
